(19) United States
(12) Patent Application Publication
(10) Pub. No.: US 2004/0166561 A1
(43) Pub. Date: Aug. 26, 2004

(54) Novel intein and uses thereof

(76) Inventors: Russell Tony Maskell Poulter, Dunedin (NZ); Timothy John Desborough Goodwin, Dargaville (NZ); Margaret Isobel Butler, Dunedin (NZ)

Correspondence Address: JACOBSON HOLMAN PLLC, 400 SEVENTH STREET N.W., SUITE 600, WASHINGTON, DC, 20004, US

(21) Appl. No.: 10/477,205

(22) PCT Filed: May 20, 2002
(86) PCT No.: PCT/NZ02/00098

(30) Foreign Application Priority Data
May 18, 2001 (NZ) 511799

(51) Int. Cl. C07H 21/04; C12N 9/50; C12N 15/74

(52) U.S. Cl. 435/69.1; 435/219; 435/252.3; 435/320.1; 536/23.2

(57) ABSTRACT

The invention provides a self-cleaving protein or intein. The intein can be obtained from a wide range of Cryptococcus neoformans. Also provided are methods of using the inteins such as in protein purification and as a target in testing the efficacy of drugs to inhibit intein function.

### TECHNICAL FIELD

The invention relates to a novel intein identified in Cryptococcus neoformans. The intein is useful as a molecular target for testing drugs to inhibit microbialfunction.

### BACKGROUND ART

Inteins are mobile protein splicing elements embedded in-frame within a precursor protein sequence and excised during protein maturation (1). Inteins have a sporadic distribution across species and proteins. They occur in all three kingdoms of life but have been found in relatively few species. Inteins that are present at the same location within homologous proteins from different organisms are termed ‘allelic’ inteins (2). Only 21 allelic inteins have been described from 14 proteins. Most inteins have been found in archea and bacteria but rarely in eukaryotes, and not in higher eukaryotes such as humans.

Most inteins have an endonuclease domain. Inteins lacking an endonuclease domain have also been identified. These “mini-inteins” account for less than 20% of all inteins and while they can still be spliced, they are not capable of spreading to new sites. To date, four non-allelic inteins have been described in eukaryotic organisms. Porphyra purpurea (a red alga) chloroplasts and Guillardia theta (a cryptomonad alga) plastids have allelic mini-intein genes in their DNA B helicase genes. The green alga Chlamydomonas eugametos has a DODendonuclease-containing intein gene in its chloroplast clp-A gene. There is an intein in the Chilo iridescent virus ribonucleotide reductase class 1 gene. An allelic intein is in the homologous gene from a Bacillus subtilis prophage (Spbeta). The only previously described nuclear eukaryote intein genes are present in the vacuolar ATPase (VMA) genes of Saccharomyces cerevisae (Sce VMA; 3) and Candida tropicalis (Ctr VMA; 4). The Sce VMA and Ctr VMA allelic inteins are of similar length (454 and 471 amino acids, respectively) and are 37% identical. Both have an endonuclease domain in addition to the splicing domains. The endonuclease of Sce VMA has been shown to cleave unoccupied target sites in the intein-less VMA genes during meiosis, resulting in ‘homing’ of the intein gene to the previously unoccupied allele (5).

Inteins have been identified as promising antimicrobial targets (U.S. Pat. No. 5,795,731 incorporated herein by reference). To be useful as a target an intein needs to be present in most or all strains of the microbe being targeted, and in a microbe which is of significant pathogenic concern. Eukaryotic inteins are also particularly useful because this group is as yet poorly characterised.

It is therefore an object of this invention to provide an intein which goes at least some way towards meeting these requirements, or at least provides the public with a useful choice.

The inventors have now unexpectedly identified a new eukaryotic intein present in a wide range of Cryptococcus neoformans. It is towards this intein (Cne PRP8) that the present invention is broadly directed.

### SUMMARY OF THE INVENTION

Accordingly, in a first aspect the present invention provides an intein identified herein as Cne PRP8 obtainable from Cryptococcus neoformans, or a functionally equivalent, or functionally altered, fragment or variant thereof.

Preferably, the intein can be isolated from C. neoformans strain Cn35 on deposit at the American Type Culture Collection, Maryland, USA under Accession No. ATCC 32045.

Conveniently, the intein is obtainable from the C. neoformans PRP8.

The intein comprises in a preferred aspect an amino acid sequence selected from the group consisting of SEQ ID NOS:1, 2, 3, 4, 5, 6, 7 and 8.

Preferably, the intein has the amino acid sequence set forth in SEQ ID NO:1, or is a functionally equivalent variant or fragment thereof.

The invention further provides an intein which is obtainable from an organism other than Cryptococcus neoformans and which is a functionally equivalent, or functionally altered, variant or fragment of an intein of the invention.

The invention also provides an isolated intein which has an amino acid sequence which has greater than about 35%, preferably greater than about 50% identity with the sequence of SEQ ID NO:1, preferably greater than about 60%, more preferably greater than about 70%, more preferably greater than about 80%, more preferably greater than about 90%, and even more preferably greater than about 95% identity with the sequence of SEQ ID NO:1.

In a further aspect, the present invention provides an isolated nucleic acid molecule encoding an intein of the invention.

The invention also provides an isolated nucleic acid molecule which encodes an intein which is part of the genome of C. neoformans strain Cn 3511, on deposit at American Type Culture Collection, Maryland, USA, under Accession No. ATCC 32045.

In a still further aspect, the present invention provides an isolated nucleic acid molecule comprising approximately 516 nucleic acids from base 142 to base 657 as set forth in FIG. 1B or fragments or variants thereof, which encode an intein of the invention.

The invention also provides an isolated nucleic acid molecule which comprises a coding sequence selected from the group consisting of SEQ ID NOS: 9, 10, 11, 12, 13, 14, 15, 16 and 17.

The nucleic acid molecules can be RNA or cDNA, but are preferably DNA molecules.

Still further, the invention provides a vector or construct, which includes a nucleic acid molecule of the invention or a fragment or variant thereof as defined above.

Hosts transformed with a vector of the invention and capable of expressing an intein of the invention are also provided.

The invention further comprises an organism, in substantially pure form, which includes a nucleic acid molecule of the invention and which is capable of expressing an intein of the invention.

In still another aspect, the present invention provides a Cne PRP8 intein or intein construct for use in medicine.

Preferably, the use is as a target for testing agents for antimicrobial activity.

The invention also provides a composition comprising an intein of the invention.

The invention in a further aspect provides a protein including an intein of the invention.

In one embodiment, the protein comprises an intein of the invention flanked by N- and C-terminal exteins.

Preferably, the N- and C-terminal exteins comprise proximal and distal extein reporter portions which together form a reporter protein.

In an alternate embodiment, the protein comprises a binding protein portion, an intein of the invention, and a reporter protein portion.

Preferably, the intein separates the binding protein portion and the reporter protein portion.

The reporter protein may be selected from an enzymatic assay protein, a protein conferring antibiotic resistance, a protein providing a direct calorimetric assay, or a protein assayable by in vivo activity.

Preferably, the reporter protein is selected from the group consisting of: thymidylate synthase, β-galactosidase, orotic acid decarboxylase, galactokinase, alkaline phosphotase, β-lactamase, luciferase, and green fluorescent protein.

In a further aspect, the invention provides a method for producing a protein, the method comprising subjecting an protein containing an intein of the invention to cleavage conditions.

In one embodiment, the protein is a fusion protein.

The invention also provides an isolated nucleic acid molecule which encodes a protein of the invention.

The invention also provides a method for screening an agent for antimicrobial activity against a microorganism, the microorganism having an intein of the invention in a gene encoding a protein which facilitates growth of the microorganism, the method comprising detecting inhibition of said intein, which comprises:

(a) preparing recombinant clones of an inducible expression vector containing: (i) an altered reporter gene comprising a silent restriction site within a reporter gene, and (ii) said intein;

(b) detecting production of extein product of said intein by said recombinant clones in the presence of said agent;

wherein reduced production of said extein product indicates inhibition of said intein, and antimicrobial activity of said agent against said microorganism.

In a further aspect, the invention provides a method for screening an agent for antimicrobial activity against a microorganism, the microorganism having an intein of the invention in a gene encoding a protein which facilitates growth of said microorganism, the method comprising detecting inhibition of said intein by monitoring intein function, which comprises:

(a) creating a silient restriction site within a reporter gene which results in an altered reporter gene;

(b) cloning said altered reporter gene into an inducible expression vector;

(c) cloning said intein into said inducible expression vector containing said altered reporter gene to generate recombinant clones; and

(d) detecting the production of extein product of said intein by said recombinant clones in the presence of said agent;

wherein reduced production of said extein product indicates inhibition of said intein, and antimicrobial activity of said agent against said microorganism.

In these methods the inteins may further comprise an additional conserved distal amino acid residue selected from cysteine, serine and threonitric.

The microorganism may be selected from a broad range of microbial pathogens, yeasts and bacteria. Preferably, the microorganism is selected from the group consisting of C. neoformans, E. coli and Saccharomyces species.

Preferably, the protein is CnePRP8

A preferred reporter gene is β-galactosidaseA preferred inducible expression vector is pUC19.

Detection of extein production is conveniently achieved by phenotype characterisation.

### DESCRIPTION OF THE DRAWINGS

While the present invention is broadly as defined above, it also includes embodiments of which the following description provides examples. In particular, a better understanding of the present invention will be gained through reference to the accompanying drawings in which:

FIG. 1 is the polynucleotide encoding the intein identified herein as Cne PRP8. The sequence is flanked by partial sequences corresponding to the extein encoding sequences. The intein coding region shown underlined begins at base 142 and ends at base 657, 516 bases inclusive. FIG. 1A is the sequence identified from strain JEC21, while FIG. 1B is the sequence isolated from strain Cn 3511. The sequences differ at three positions, bases 84, 129 and 147 (in bold).

FIG. 2 is the partial sequence of PRP8 protein precursor (from 3511). The amino acid sequence is a translation of the DNA sequence described in FIG. 1B). The intein sequence is underlined. Residues which are highly conserved in a wide range of inteins are marked *. The residue immediately after the intein (in the C-terminal extein) is also highly conserved and is marked #. The intein begins at residue 48 (C) and ends at residue 219 (N), 172 amino acids inclusive.

FIG. 3 is a partial identity comparison between the PRP8 sequence of C. neoformans and the PRP8 sequences of other related organisms showing an in-frame insertion in the C. neoformans sequence (corresponding to the intein). This begins with a C and ends with an N, and is 172 amino acids in length. The GenBank accession numbers for the PRP8 sequences are as follows: A. thaliana, AAD55467; C. elegans, AAA27977; D. melanogaster, AAF58573; H. sapiens, AAC61776; S. cerevisiae, AAB68011; S. pombe, CAB11062. The insertion was not found in other organisms, particularly humans.

FIG. 4 is an alignment of the N-terminal end of Cne PRP8 with the N-terminal ends of the VMA inteins of C. tropicalis and S. cerevisae. The completely conserved residues (darkest shading) include the catalytically critical N-terminal cysteine (C).

FIG. 5 is an alignment of the C-terminal end of the of Cne PRP8 with the C-terminal ends of the VMA inteins of C. tropicalis and S. cerevisae. The completely conserved residues (darkest shading) include the catalytically critical C-terminal HN motif.

FIG. 6 is a multiple DNA sequence alignment of the CnePRP8 intein coding sequences from Cryptococcus neoformans species.

FIG. 7 is a multiple amino acid sequence alignment of the Cne PR8 inteins from Cryptococcus neoformans.

### DESCRIPTION OF THE INVENTION

As broadly outlined above, in one aspect the present invention provides a novel intein.

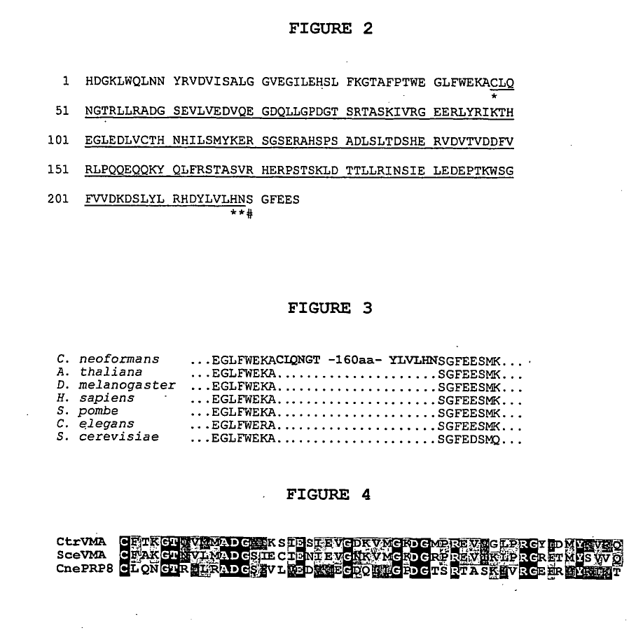

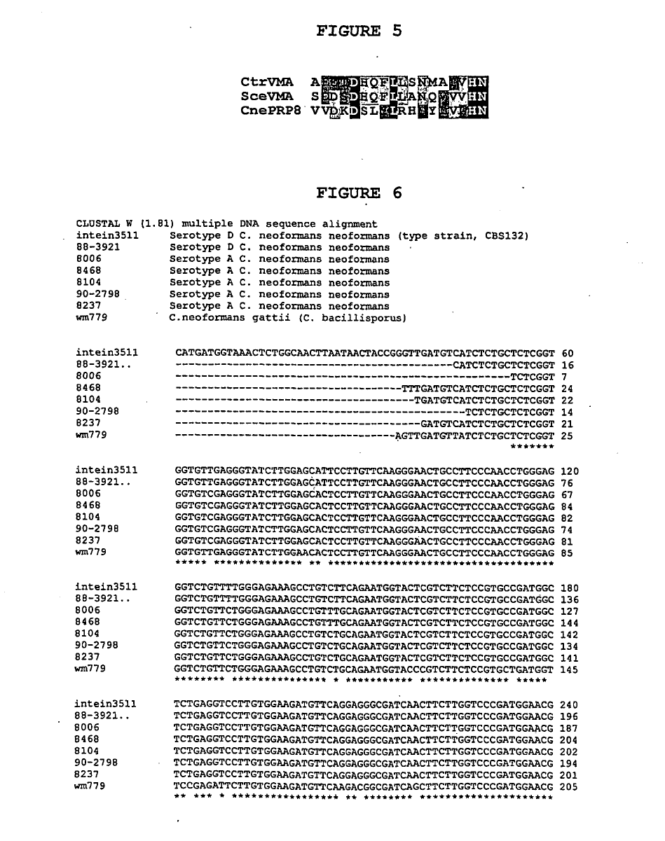

The intein identified as Cne PRP8 comprises the amino acid sequence set forth in FIG. 2 (bases 48 to 219), and SEQ ID NO:1. Conveniently, Cne PRP8 is obtainable from the C. neoformans PRP8. Functionally equivalent variants are also contemplated. To date, functionally equivalent variants have been identified in 47 C. neoformans strains indicating the intein is widespread across the species. Conveniently, an intein of the invention may be obtained from C. neoformans strain Cn3511, on deposit with American Type Culture Collection, Maryland, USA, under deposit number ATCC 32045.

Cne PRP8 is the first intein to be derived from a basidiomycete and is only the second (non-allelic) eukaryotic nuclear intein to be identified. As the intein coding sequence does not appear to encode a central endonuclease, Cne PRP8 is deemed to be a ‘mini-intein’.

The intein of the invention can include its entire amino acid sequence or can include only parts of that sequence where such parts constitute active fragments. Such activity will be as an intein.

Extended inteins which include a conserved residue distal to the intein are also provided. The conserved residue immediately distal to the intein may be selected from the group consisting of cysteine, serine and threonine.

One extended intein for use in the invention has the amino acid sequence:

```
                                                                            ACLQ
                                                                              *
NGTRLLRADG SEVLVEDVQE GDQLLGPDGT SRTASKIVRG
EERLYRIKTH EGLEDLVCTH NHILSMYKER SGSERAHSPS
ADLSLTDSHE RVDVTVDDFV RLPQQEQQKY QLFRSTASVR
HERPSTSKLD TTLLRINSIE LEDEPTKWSG FVVDKDSLYL
RHDYLVLHNS
            **#
```

As noted above, the invention also includes within its scope functionally equivalent variants of the intein of SEQ ID NO:1. Seven functionally equivalent variants are depicted in FIG. 7 and (SEQ ID NOS:2 to 8). These are only examples of variants, and variants are not limited to these sequences.

The phrase “functionally equivalent variants” recognises that it is possible to vary the amino acid of a protein while retaining substantially equivalent functionality. For example, a protein can be considered a functional equivalent of another protein for a specific function if the equivalent peptide is immunologically cross-reactive with and has at least substantially the same function as the original intein.

Functionally altered variants are also contemplated. It will be appreciated by the skilled reader that highly conserved sequences appear at the junction of inteins and exteins (see InBase http://www.ineb.com/inteins). Serine(s), Threonine(T) or Cysteine (C) occur at the N-terminal end, while Histidine (H) and Asparagine (N) occur at the C-terminal end of the intein. It is recognised in the art that mutation or deletion of these amino acid residues alters intein function for example to yield cleavage at one or both of the intein-extein junctions, or to result in an intein incapable of splicing or cleavage. (See Chong et al. (1998b) J. Biol. Chem. 273:10567-10577, and WO 01/12820). The term “functionally altered” as used herein includes all such inteins.

The functionally equivalent or altered protein need not be the same size as the original. The equivalent or altered protein can be, for example, a fragment of the protein, a fusion of the protein with another protein or carrier, or a fusion of a fragment with additional amino acids. Active fragments may be obtained by deletion of one or more amino acid residues of full length Cne PRP8. It is also possible to substitute amino acids in a sequence with equivalent amino acids using conventional techniques. Groups of amino acids normally held to be equivalent are:

(a) Ala, Ser, Thr, Pro, Gly;

(b) Asn, Asp, Glu, Gln;

(c) His, Arg, Lys;

(d) Met, Leu, Ile, Val; and

(e) Phe, Tyr, Trp.

That equivalent may, for example, be a fragment of the intein containing, for example, from 165 to 171 amino acids, a substitution, addition, or deletion mutant of the intein, or a fusion of the protein or a fragment or a mutant with other amino acids.

Polypeptide sequences may be aligned, and percentage of identical amino acids in a specified region may be determined against another sequence, using computer algorithms that are publicly available. The similarity of polypeptide sequences may be examined using the BLASTP algorithm. BLASTP software is available on the NCBI anonymous FTP server (ftp://ncbi.nlm.nih.gov) under /blast/executables/. The use of the BLAST family of algorithms, including BLASTP, is described at NCBI's website at URL http://www.ncbi.nlm.nih.gov/BLAST/newblast.html and in the publication of Altschul, Stephen F., et al. (1997), “Gapped BLAST and PSI-BLAST: a new generation of protein database search programs”, Nucleic Acids Res. 25:3389-34023.

Accordingly, in a further aspect, the invention also provides an intein isolatable from C. neoformans, which includes one or more active peptides from within the amino acid sequence set forth in FIG. 2.

A specific mini-intein of the invention identified has the following amino acid sequence:

| | |
|---|---|
| CLQNGTRLLRADG | (SEQ ID NO:1) |
| SEVLVEDVQEGDQLLGPDGT | |
| SRTASKIVRGEERLYRIKTH | |
| EGLEDLVCTHNHILSMYKER | |
| SGSERAHSPSADLSLTDSHE | |
| RVDVTVDDFVRLPQQEQQKY | |
| QLFRSTASVRHERPSTSKLD | |
| TTLLRINSIELEDEPTKWSG | |
| FVVDKDSLYLRHDYLVLHN | |

or a functionally equivalent variant or fragment thereof.

Polypeptides of the invention also include homologous polypeptides having an amino acid sequence with about 35%, preferably about 50%, preferably at least 60%, more preferably at least 70% identity to the intein of the invention, preferably at least about 80% identity, more preferably at least about 90% identity, as well as those polypeptides having an amino acid sequence at least about 95% identical to the intein.

An intein of the invention together with its active fragments and other variants may be generated by synthetic or recombinant means. Synthetic polypeptides having fewer than about 100 amino acids, and generally fewer than about 50 amino acids, may be generated by techniques well known to those of ordinary skill in the art. For example, such peptides may be synthesised using any of the commercially available solid-phase techniques such as the Merryfield solid phase synthesis method, where amino acids are sequentially added to a growing amino acid chain (see Merryfield, J. Am. Chem. Soc 85: 2146-2149 (1963)). Equipment for automative synthesis of peptides is commercially available from suppliers such as Perkin Elmer/Applied Biosystems, Inc. and may be operated according to the manufacturers instructions.

Fragments may be obtained by deletion of one or more amino acid residues of the full length intein. This may be by stepwise deletion of amino acid residues, from the N- or C-terminal end of the intein, or from within the intein.

An intein of the invention, or a fragment or variant thereof, may also be produced recombinantly by inserting a polynucleotide (usually DNA) sequence that encodes the protein into an expression vector and expressing the protein in an appropriate host. Any of a variety of expression vectors known to those of ordinary skill in the art may be employed and include plasmids, pUC and pET serves plasmids, particularly pUC19. Expression may be achieved in any appropriate host cell that has been transformed or transfected with an expression vector containing a DNA molecule which encodes the recombinant protein. Suitable host cells includes procaryotes, yeasts and higher eukaryotic cells. Preferably, the host cells employed are E. coli, yeasts or a mammalian cell line such as COS or CHO, or an insect cell line, such as SF9, using a baculovirus expression vector. E. coli and Sacchromyces species are particularly preferred. C. neoformans and other Cryptococcus species may of course also be used as host cells. The DNA sequence expressed in this manner may encode the naturally occurringintein, fragments of the naturally occurring protein or variants thereof.

Variants of the intein may also be prepared using standard mutagenesis techniques such as oligonucleotide-directed site specific mutagenesis.

In a further aspect, the invention also provides an organism, in substantially pure form which includes a nucleic acid molecule which is capable of expressing an intein of the invention. In a preferred embodiment, the organism is recombinantly produced, and may be selected from E. coli and Saccharomyces species.

DNA sequences encoding the intein or fragments may be obtained by screening an appropriate C. neoformans cDNA or genomic DNA library for DNA sequences that hybridise to degenerate oligonucleotides derived from partial amino acid sequences of the protein. Suitable degenerate oligonucleotides may be designed and synthesised by standard techniques and the screen may be performed as described, for example, in Maniatis et al. Molecular Cloning—A Laboratory Manual, Cold Spring Harbour Laboratories, Cold Spring Harbour, N.Y. (1989), Sambrook et al (12). The polymerase chain reaction (PCR) may be employed to isolate a nucleic acid probe from genomic DNA, a cDNA or genomic DNA library. The library screen may then be performed using the isolated probe.

In a yet further aspect, the invention provides an isolated nucleic acid molecule encoding an intein of the invention.

A specific nucleic acid molecule of the invention includes the following nucleotide sequence:

| | | |
|---|---|---|
| 1 | TGTCTTCAGA ATGGTACTCG TCTTCTCCGT GCCGATGGCT CTGAGGTCCT | (SEQ ID NO:9) |
| 51 | TGTGGAAGAT GTTCAGGAGG GCGATCAACT TCTTGGTCCC GATGGAACGA | |
| 101 | GCAGGACGGC GAGCAAGATT GTTCGCGGCG AAGAGCGTCT CTACCGTATC | |
| 151 | AAAACCCATG AGGGGCTCGA AGATCTTGTC TGTACCCATA ACCACATCCT | |
| 201 | TTCTATGTAT AAAGAAAGGT CTGGTTCGGA GCGAGCTCAT TCTCCTAGTG | |
| 251 | CCGACCTCAG CCTCACAGAC AGCCATGAGA GAGTCGATGT GACTGTCGAT | |
| 301 | GACTTTGTCC GCCTTCCTCA ACAAGAGCAA CAGAAGTATC AGCTTTTCCG | |
| 351 | TTCAACTGCT TCTGTGCGAC ACGAGCGACC ATCCACTTCT AAATTAGACA | |
| 401 | CCACCTTGTT ACGCATCAAT TCTATCGAGC TTGAGGACGA GCCTACGAAG | |
| 451 | TGGTCCGGTT TTGTGGTTGA CAAAGACAGT CTTTATCTTC GTCATGACTA | |
| 501 | TTTGGTATTG CACAAC | |

or a fragment or variant thereof.

The invention also includes within its scope, homologues or variants of an isolated nucleic acid molecule encoding an intein of the invention. Specifically contemplated are allelic variants, which occur even though they have low (under 50%) percentage identity. Polynucleotide sequences may be aligned, and percentage of identical nucleotides in a specified region may be determined against another sequence, using computer algorithms that are publicly available.

Two exemplary algorithms for aligning and identifying the similarity of polynucleotide sequences are the BLASTN and FASTA algorithms. The BLASTN software is available on the NCBI anonymous FTP server (ftp://ncbi.nlm.nih.gov) under /blast/executables/. The BLASTN algorithm version 2.0.4 [Feb. 24, 1998], set to the default parameters described in the documentation and distributed with the algorithm, is preferred for use in the determination of variants according to the present invention. The use of the BLAST family of algorithms, including BLASTN, is described at NCBI's website at URL http://www.ncbi.nlm.nih.gov/BLAST/newblast.html and in the publication of Altschul, Stephen F, et al (1997). “Gapped BLAST and PSI-BLAST: a new generation of protein database search programs”, Nucleic Acids Res. 25:3389-3402. The computer algorithm FASTA is available on the Internet at the ftp site ftp://ftp.virginia.edu.pub/fasta/. Version 2.0u4, February 1996, set to the default parameters described in the documentation and distributed with the algorithm, is preferred for use in the determination of variants according to the present invention. The use of the FASTA algorithm is described in the W R Pearson and D. J. Lipman, “Improved Tools for Biological Sequence Analysis,” Proc. Natl. Acad Sci. USA 85:2444-2448 (1988) and W. R. Pearson, “Rapid and Sensitive Sequence Comparison with FASTP and FASTA,” Methods in Enzymology 183:63-98 (1990).

All sequences identified as above qualify as “variants” as that term is used herein.

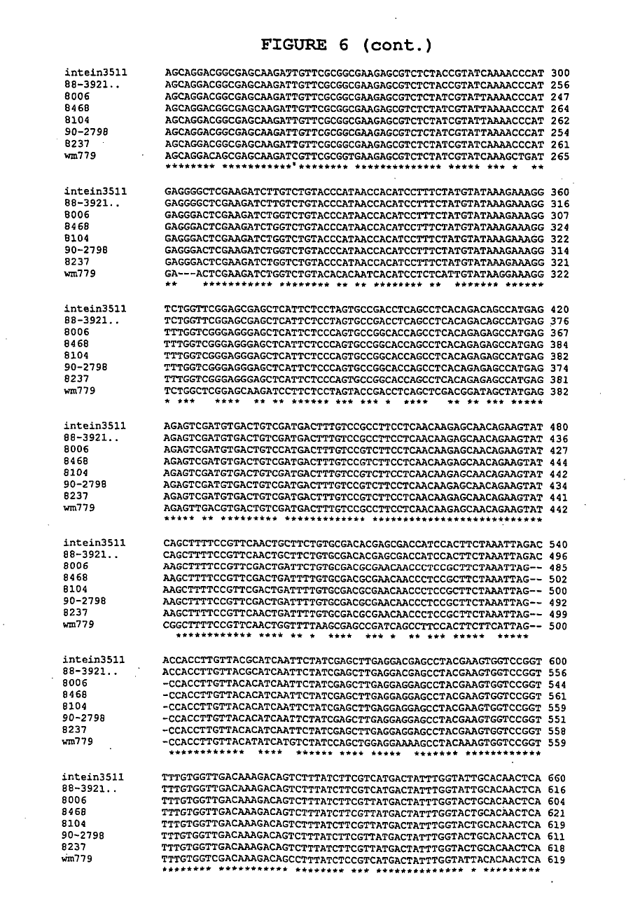

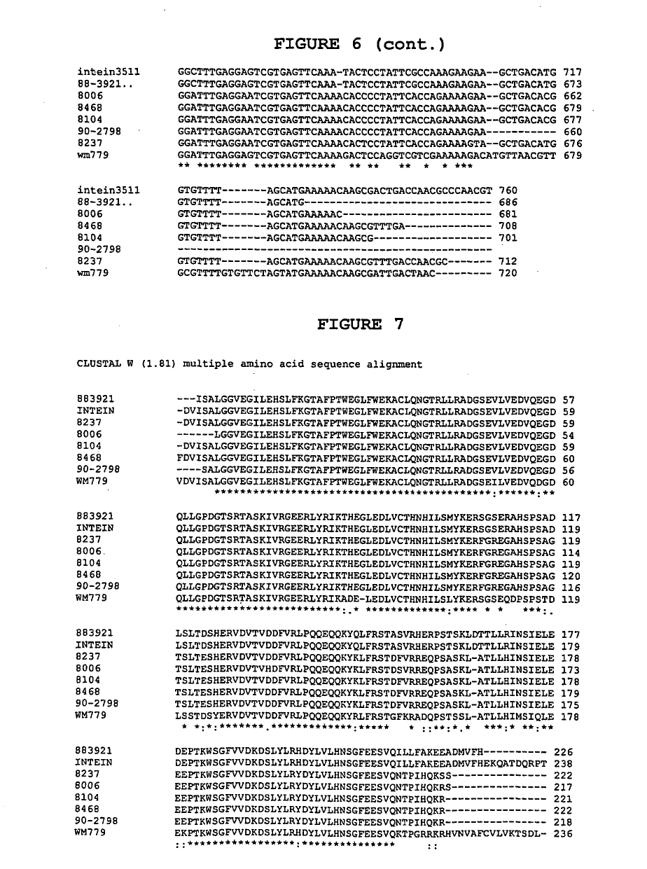

The invention also includes nucleic acid molecules or polynucleotides that comprise a polynucleotide sequence encoding an intein having at least about 35% identity, preferably at least about 50%, preferably at least about 60%, preferably at least about 70% identity, preferably at least about 85% identity, more preferably at least about 90% identity, as well as those polynucleotides having a nucleic acid sequence at least about 95%, 97%, 98%, or 99% identical to the intein nucleotide sequence of SEQ ID NO:1. Nucleotide sequences encoding inteins which are variants of the intein of the invention have been located for the first time by the applicant in 47 strains of C. neoformans identified in Table 1 below). Exemplary variant sequences are set forth in FIG. 6, and SEQ ID NOs:9 to 17.

An intein, intein fragment or nucleic acid molecule of the invention may be generated by synthetic or recombinant means by techniques well known to those of ordinary skill in the art. Variants may be prepared using standard mutagenesis techniques or may be isolated from organisms.

Variant polynucleotide sequences will generally hybridize to the polynucleotide sequence under stringent conditions. This term will be recognised by those skilled in the art and is discussed in Maniatis et al. and Sambrook et al; (supra). As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C. Such hybridizable sequences include those which code for the equivalent intein from sources (such as other Cryptococcus) other than Cryptococcus neoformans.

While synthetic or recombinant approaches can be employed, it is however practicable (and indeed presently preferred) to obtain the Cne PRP8 sequence by isolation from the C. neoformans PRP8 gene with C. neoformans strain ATCC 32045 being presently preferred. The host gene, PRP8, encodes a highly conserved mRNA splicing protein found as the core of the spliceosome.

Once obtained, the intein is readily purified if desired. This may involve affinity chromatography. Other approaches to purification (e.g. gel-filtration or anion exchange chromatography) can also be used. Where the intein or fragment is produced in the form of a fusion protein, the carrier portion of the fusion protein can prove useful in this regard.

Furthermore, if viewed as desirable, additional purification steps can be employed using approaches which are standard in the art. The approaches are fully able to deliver a highly pure preparation of the intein. Preferably, the intein preparation comprises at least about 50% by weight of the protein, preferably at least about 80%, preferably at least about 90%, and more preferably at least about 95% by weight of the protein.

The purification procedure will of course depend on the degree of purity required for the use to which the intein, fusion protein or fragment is to be put.

Once obtained, the intein and/or its fragments and/or its functionally equivalent variants can be formulated into a composition. The composition can be, for example, a therapeutic composition for application as a veterinary, pharmaceutical, or diagnostic composition. For these purposes it is generally preferred that the intein be present in a pure or substantially pure form. Again, standard approaches can be taken in formulating such compositions (see, for example, Remington's Pharmaceutical Sciences, 18^{th} Edition, Mack Publishing (1990)).

The inteins and proteins and/or primers therefore of the present invention can also be included in assay kits. Polymerase chain reactions using appropriate primers flanking the intein may be used to diagnose infection. The kit may further include PCR primers, thermostable polymerase, deoxyribose triphosphates, and buffer. The assay carried out may be by RealTime PCR or by agarose gel electrophoresis detecting an amplified DNA band of the appropriate size. The PCR product could be subjected to DNA sequencing to identify specific types or strains.

In a further aspect, the invention also provides a process for producing a protein of interest, particularly fusion proteins comprising a protein of interest and an intein of the invention. The intein may, for example, be used in conjunction with an affinity group to purify a desired protein. Affinity fusion-based protein purification is taught, for example in Chong et al. (1997b) Gene 192: 271-281; Chong et al. (1998b) Nucl. Acids Res. 26: 5109-5115, and WO 01/12820 all incorporated herein by reference. Where self-cleavage of the intein occurs, rather than splicing the desired protein is released without the need for protease addition, simplifying purification. Cleavage may be achieved using standard art protocols by blocking the later stages of intein splicing or using an intein mutant in one of the amino acids critical for completion of splicing. For example mutants lacking the conserved N-terminal and C-terminal residues as discussed above. It is likely that conditions favouring cleavage of the PRP8 intein will differ from others such as the VMA intein, these points of difference may be usefully employed.

The strategies are usefully represented as follows:

Splicing Nₓ−I−Cₓ where Nₓ and Cₓ are the proximal and distal exteins respectively and I is an intein of the invention, splice to form NₓCₓ+I

Cleavage Nₓ−I−Cₓ cleave to form NₓI+Cₓ

The N- and C-terminal flanking exteins may be from the same protein (cis splicing or cleavage), or from different proteins (trans splicing or cleavage).

In one embodiment, the N- and C-terminal proximal and distal exteins comprise the protein C. neoformans PRP8.

In an alternate embodiment, the N- and C-terminal proximal and distal exteins comprise a receptor protein.

The protein or fusion protein may as discussed above be produced by chemical synthesis, or recombinantly or according to other known art techniques. Preferably, the protein is produced recombinantly by preparing a vector containing nucleic acid sequences and/or DNA encoding the protein or fusion protein, transforming a host cell with the vector, and expressing the nucleic acid/DNA in the host cell. Vectors and host cells as discussed may be employed.

The protein produced may be purified as described above, using standard art techniques.

The invention also provides a method for purifying a protein of interest. The method comprises producing a fusion polypeptide comprising a binding protein portion, an intein of the invention and a protein of interest portion, binding the fusion polypeptide to a binding moiety, subjecting the intein to cleavage conditions, and separating the desired protein. Binding may comprise binding of the fusion polypeptide to an affinity matrix (e.g. beads, membranes, columns, or material in a column). Separation can include subjecting the matrix (e.g. column contents) to a chemical or physical change such as pH or temperature shift, and eluting the desired protein. Useful cleavage conditions are known in the art for example in WO 01/12820 incorporated herein by reference.

In the situation where the protein is synthesised as a protein of interest/intein/binding moiety fusion, and the binding moiety is recognised by and retained on a column, a one-step purification method is feasible. For example, if the protein of interest is fused to an intein with a distal chitin binding domain the tripartite protein may be bound to a chitin column in the presence of zinc. Elution of the column with EDTA will chelate the zinc and allow the intein to cleave from the protein of interest. The intein can be modified so as to prevent the splicing reaction. The result is that the protein of interests elutes from the column while the intein and chitin binding domain remain. Tripartite fusions and purification methods are discussed in WO 01/12820 above. Cleavage is also achieved as described above.

In one embodiment, the protein of interest portion is a reporter protein portion. The intein may separate the binding protein portion and the reporter protein portion, or protein of interest portion.

The binding portion may for example be maltose binding protein of E. coli or a His-tag.

Accordingly, in a further aspect, the invention also provides a protein including an intein of the invention. Preferred proteins include those with N- and C-teminal proximal and distal exteins, as well as binding protein/intein/reporter protein fusion proteins as discussed above. It will also be appreciated that in some cases the protein is a precursor protein produced prior to intein splicing or cleavage. The invention further provides isolated nucleic acid molecules which encode the proteins of the invention. These nucleic acid molecules may be produced according to the methods discussed above.

As noted above, the invention also has application in screening agents for antimicrobial activity against a microorganism. In this method, an intein of the invention is present in a gene encoding a protein which facilitates growth of a microorganism. The microorganism may be selected from a broad range of microbial pathogens such as Candida and Cryptococcus; yeasts such as Saccharomyces; and bacteria such as E. coli. Preferably, the microorganism is selected from the group consisting of C. neoformans, E. coli, and Saccharomyces species. E. coli is particularly useful to facilitate intial screening. Screening requires the preparation of an inducible expression vector containing an altered reporter gene which contains a silent restriction site therein, and the intein of the invention therein. The vector is expressed in a host cell. Production of extein product of the intein is detected and/or measured in the presence of an agent of interest.

A reduction in the amount of extein produced indicates that the intein has been inhibited, and that the agent has inhibitory activity against the intein. From this it may be reasonably inferred that the agent may inhibit the growth of a microorganism incorporating the intein, particularly natural pathogens. The agent tested may be employed in the screening methods of the invention at varying concentrations. In this way the most effective concentrations of the agent can also be determined.

More specifically, the present invention relates to a genetic system to monitor intein function based on the cloning of the Cryptococcus neoformans PRP8 intein (CnePRP8) into the α peptide of the β-galactosidase of E.coli. This may conveniently be in a plasmid such as pUC19. The uninterrupted α peptide, which encodes the amino fragment of the β-galactosidase, has been developed as a cloning reporter gene. The uninterrupted α peptide gene can complement E. coli cells deficient in β-galactosidase for growth on medium with lactose as a sole carbon source (minimalMedium+lactose), conferring a Lac+phenotype. In contrast, if sequences are inserted within the α peptide coding sequence they may interfere with translation or the activity of the peptide. In-frame fusions of the Cne PRP8 intein will be made to the α peptide exteins. If bacteria carrying this construct are Lac+this indicates the α peptide sequence is active, the activity of the enzyme indicating that the intervening protein sequence (the intein) has been excised from the precursor protein.

Preferably, the protein is Cne PRP8. When the protein is Cne PRP8 cloned into pUC19, the method results in the production of clones pCCne (active intein) and PRCne (inactive intein).

Similarly, a wide range of reporter genes/proteins may be employed in the proteins, protein preparation, protein purification and screening methods of the present invention. Preferred reporter genes/proteins are easily assayable either in vivo or in vitro, or both, and include but are not limited to β-galactosidase, galactokinase, luciferase, alkaline phosphotase (for enzymatic assays), β-lactamase (a reporter conferring antibiotic resistance), orotic acid decarboxylase and green fluorescent protein, a reporter useful in direct colormetric assays.

β-galactosidase is particularly preferred for use. The presence of extein is readily measured by spectrophotometric assays using this enzyme. Alternatively it can be assayed in vivo by liquid growth assay of bacteria in minimal medium+lactase (turbidity) or on petri plates (using various synthetic galactosides).

Detection of extein product may be achieved by standard analytical methods such as phenotype characterisation, protein characterisation, for example by amino terminal sequence mapping of tryptic peptides and mass spectroscopy enzyme assays, and colorimetric methods all of which are well known to those versed in the art.

As will be appreciated using this method a precursor protein is synthesized comprising exteins interrupted by an intein. Protein splicing then results in intein excision, and extein ligation, which restores the uninterrupted reading frame to the intein-containing protein. Highly conserved sequences appear at the junction of the inteins and the exteins. Ser (S), Thr (T) or Cys (C) occur at the N-terminal end while His (H) and Asn (N) occur at the C-terminal end of the intein. In addition there is a highly conserved extein residue immediately adjacent to the C-terminal Asn of the intein, either a Cys, Thr or Ser.

The presence of these conserved amino-acids is employed in the detection of inteins in genomic sequence. An intein presents as an in phase insertion in the coding sequence of a gene, there should be homologues of the gene which lack the intein, the intein will display the characteristic N-terminal and C-terminal amino acids and there will be some degree of protein sequence homology to other inteins.

The invention will now be described more fully in the following experimental section, which is provided for illustrative purposes only.

### EXAMPLE 1

### Materials and Methods

Host Cryptococcus neoformans strain Cn 3511 (ATCC 32045) obtained from the Intstitute of Environmetal Science & Research Ltd, Porirua, New Zealand.

Host Culture: Strain Cn 3511 was grown at 27° C. in YPD medium (1% w/w Dyco Yeast extract, 2% w/v peptone, 2% w/w glucose, solidified with 1.5% w/v agar when necessary).

DNA Isolation: Genomic DNA was isolated from 50ml overnight cultures essentially via the method of Philipson et al. (7)

Amplification of the intein sequence and flanking nucleotide regions was accomplished with the Expand High Fidelity PCR system (Roche) using the following primers (Genset, Singapore): FcnIn, 5′ gcggaattcCCACATGGTGAATCGACG and CnInR, 5′ gctctagaTCATCTGGACTAACCAGC, at a final concentration of 1 uM. Approximately 100 ng of genomic DNA were used per 100 ul reaction. The Polymerase Chain Reaction (PCR) annealing temperature was 52° C. (1 minute) with extension at 72° C. (2 minutes). The resulting 821 bp PCR product was purified with a Qiagen column prior to automatic sequencing. Both strands of the PCR product were sequenced using an ABI 377DNA Sequencer. The consensus intein sequence for strain Cn3511 was generated using EditView 1.0.1 (Perkin Elmer). Sequence analyses were conducted using BLAST programs available at NCBI (http://ww.ncbi.nlm.nih.gov/blast/) and via the Stanford Cryptococcus neoformans genome sequencing project webstite (http://baggage.stanford.edu/cgimisc/cneoformans/cneo_blast.cgi). Multiple alignments were generated with Clustal X (8) edited with Seaview (9) and shaded with MacBoxshade (http://www.isrec.isb-sib.ch/sib-isrec/boxshade/macBoxshade).

### Intein Identification

An intein gene was identified in Cryptococcus neoformans by performing a TBLASTN search of the C. neoformans sequence database (http://baggage.stanford.edu/cgimisc/cneoformans/cneo_blast.cgi) using the amino acid sequence of the C. tropicalis intein (Ctr VMA) as a query. The intein gene is present in the C.neoformans PRP8 gene.

Intein: The Cryptococcus neoformans intein sequence in the PRP8 gene (Cne PRP8) and flanking sequences have been assigned GenBank accession number AF349436 has been added to the InBase records (http://www.neb.com.com/neb/inteins.html).

### Results

The mini-intein, Cne PRP8 was located in the C. neoformans PRP8 gene. With reference to FIGS. 2 and 3, Cne PRP8 characteristically begins with a Cysteine residue, ends with a dipeptide Histidine-Asparagine and immediately is followed by a Serine residue. The intein contains no recognizable endonuclease domain but is similar in sequence at the N-terminus and C-terminus to the previously identified yeast nuclear inteins (FIGS. 4 & 5).

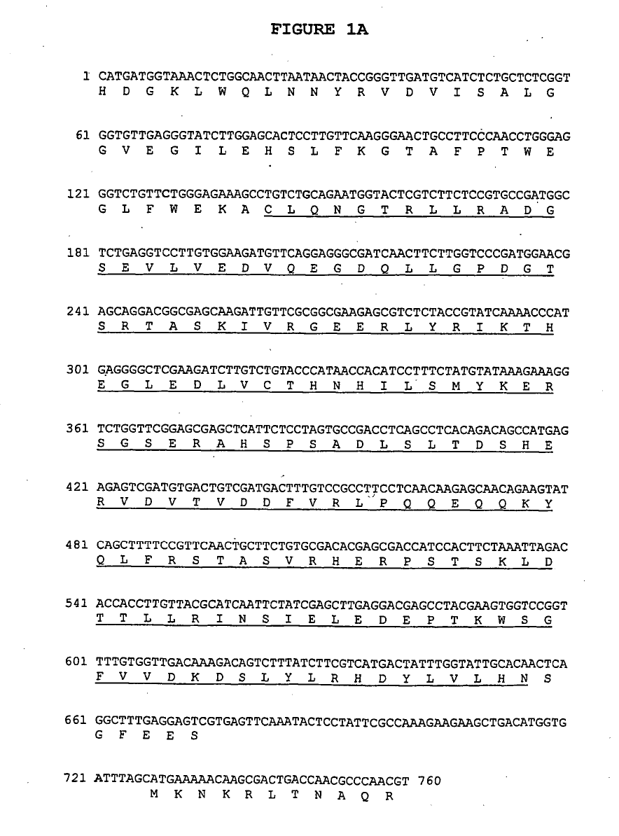

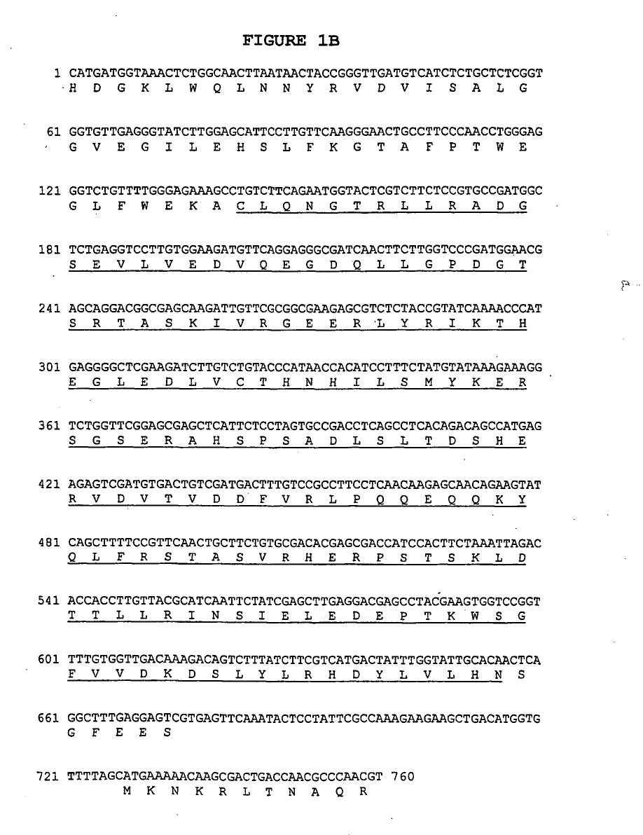

A mini-intein comprising 516 base pairs (FIG. 1A) was identified in the C. neoformans sequence database (generated from strain JEC21). Using this information, PCR primers were designed to amplify Cne PRP8 and its flanking sequences from another strain of C. neoformans, Cn 3511. 760 based pairs of sequence data were obtained from Cn 3511, in which the same mini-intein nucleic acid molecule was identified (FIG. 1B).

The protein splicing elements produced a conceptual translation of the two intein nucleic acid sequences (Cne PRP8-JEC21 and Cne PRP8-Cn3511) were too short to encode a “full-length” intein having an endonuclease domain. The Cne PRP8-Cn3511 sequence matched almost exactly the corresponding sequence of Cne PRP8-JEC21 derived from the Cryptococcus database. There were only three base differences between the DNA sequences of the two strains, only one within the intein sequence. All three differences are located at the third base codon position leading to identical predicted PRP8 amino acid sequences. The result indicated a high degree of DNA sequence identity (99.8%) between the intein sequence of the two strains of C. neoformans Cn3511 and JEC21.

### Discussion

BLASTN searches at the Cryptococcus database with the Cn 3511 polynucleotide (FIG. 1A) indicate only one significant match to a sequence present in contig 7530 (Cryptococcus database, January 2001). While the Cryptococcus genome sequence data is not yet complete, this suggests that there is probably only a single copy of the intein present in the haploid genome of JEC21. This indicates that there are probably no non-allelic versions of the intein elsewhere in the genome of Cryptococcus.

BLASTX searches of the GenBank database, with contig 7530 from the Cryptococcus database as the query, found highly significant (E=0) matches to the PRP8 gene of several species, including Homo sapiens, Trypanosoma brucei, Arabidopsis thaliana and Schizosaccharomyces pombe (not shown). These matches, however, do not include the region of the C. neoformans sequence containing the intein (FIG. 3). Of the PRP8 gene sequences available for three yeast species, Candida albicans, S. cerevisiae and S. pombe, none appear to have an intein in their PRP8 homologue (data not shown). The GenBank database carries a wide range of highly similar sequences from other eukaryote species that describe PRP8 homologues (For example, Trichomonas, Giardia, Guillardia, Plasmodium, Mus and related organisms listed in FIG. 3). None of these PRP8 homologues appear to contain an intein. The fact that the Cne PRP8 intein coding sequence is present in the PRP8 gene product, which is involved in mRNA splicing (10), suggests that it is located in the nucleus. As such, Cne PRP8 is the second, non-allelic, eukaryote nuclear intein gene to be identified. Cne PRP8 is the first intein detected in any PRP8 gene. Cne PRP8 is also the first intein to be found in a Basidiomycete. The intein is almost certain to still be functional, that is, capable of protein splicing. If it could not be auto-catalytically removed, the resulting defective PRP8 gene product would almost certainly be inactive, resulting in the Cryptococcus being unable to process pre-mRNA (a lethal situation).

BLAST searches at InBase (http://vent.neb.com) using the Cne PRP8 intein protein sequence as the query found 7 matches where the E-value was less than 0.1. As with the GenBank search, the most significant E-values were with the allelic VMA inteins of C. tropicalis and S. cerevisiae. The intein in the DNA polymerase (alpha family) gene of the three archaeans Thermococcus aggregans, T. fumicolans and Pyrococcus kodakaraenisis show matches (E=0.0008, E=0.016, E=0.067, respectively) to the first approximately 50 amino acids of Cne PRP8, and in addition have a less significant match to an internal 50 amino acid sequence. All three archaeal inteins contain endonuclease domains and there are apparently no mini-inteins in homologous alpha family DNA polymerases (Pietrokovski, S., http://bioinfor.weizmann.ac.il/˜pietro/inteins/).

BLAST searches at GenBank with the amino acid sequence of the C. neoformans intein (FIG. 2) reveal significant matches to Ctr VMA and the first 50 residues of Sce VMA (E=1e-04, E=2e-04 respectively). Further examination shows that the Cne PRP8, Ctr VMA and Sce VMA inteins share regions of similarity at both ends, perhaps reflecting a conserved self-splicing function in Cne PRP8 (FIGS. 4 & 5). Cne PRP8 is only 172 amino acids long, which is substantially shorter than the S. cerevisiae and C. tropicalis VMA inteins. This is because Cne PRP8 lacks the endonuclease domain found in the other yeast nuclear inteins.

There is no evidence, as yet, for the presence in Cryptococcus of a full-length intein encoding an endonuclease domain (not shown). Many bacterial, archaeal and plastid inteins also lack apparent endonuclases. These mini-inteins, which account for approximately 18% of all inteins, may represent defective versions of larger inteins. The derivative mini-inteins can still be spliced, but are no longer capable of spreading to new sites. Alternatively, mini-inteins may represent the ancestral form of the intein, prior to invasion by an endonuclease (Pietrokovski, http://bioinfo. Weizmann.ac.il/˜pietro/inteins/).

### EXAMPLE 2

### Strains

The strategy of using the intein as a drug target will only be effective if the intein is present in most if not all strains. The PRP8 sequences flanking the intein are highly conserved and therefore represent a conserved template for primer design and PCR amplification (Butler Mich., Goodwin T J, Poulter R T. (2001) A nuclear-encoded intein in the fungal pathogen Cryptococcus neoformans. Yeast. 18(15):1365-70).

We have amplified the Cryptococcus neoformans intein-encoding sequences from the 47 strains tested (see Table 1 below PHLS stands for Public Health Laboratory Service. IUM stands for Istituto di Igiene e Medicina Preventiva, Universita degli Studi, IRCCS Milan), suggesting that all C. neoformans strains carry the intein. These sequences have been detected as insertions in the conserved sequences of the PRP8 gene of Cryptococcus. No other organism has been reported to contain an intein encoding sequence in this gene (PRP8). The amplified sequence is of a characteristic size (˜820 bp) as estimated on agarose gels.

We have amplified the intein from 10 strains of serotype A and 10 strains of serotype D, which were originally isolated in Italy. In addition, 12 clinical isolates from Thailand, Uganda and the U.K have all yielded intein sequences; all are most similar to serotype A, which is by far the predominant strain in AIDS patients. As well as four further C. neoformans var. neoformans strains isolated in Australia, we have recently received isolates of Cryptococcus neoformans var. gattii (also termed Cryptococcus bacillisporus) from Dr Weiland Meyer (Westmead Hospital in Sydney). These C. n. var gattii are serotype B and C strains, which cause disease in immunocompetent hosts. We have shown that all 8 strains of C. n. var. gattii have an intein encoding sequence in the PRP8 gene. We have one fully sequenced C.n. var gattii intein encoding sequence.

We have further characterised a subset (15) of these intein encoding insertions in PRP8 by sequence analysis. We have integrated this information with sequence data available on the public databases: strain JEC21/serotype D: http://www-sequence.stanford.edu/group/C.neoformans/index.html strain H99/serotype A: http://cneo.genetics.duke.edu/

There were consistent differences in the intein-encoding DNA sequence between serotype A (the predominant type in AIDS patients) and serotype D strains, and further differences between these and the one C. var gattii strain fully sequenced. This variation is more apparent in some regions of the sequence than in others, particularly in the intron.

Conceptual translation of the sequences from the amplified PCR products confirms that they represent a region of the PRP8 gene with an intron of ˜52 base pairs and, in addition, an insertion not found in other PRP8 genes. This characteristic insertion is the intein encoding sequence. It contains an open reading frame (in the same frame as the PRP8 gene) and conserved residues which are characteristic of inteins (see FIG. 1a, 1b and 2).

We have aligned a representative sample of both the DNA and protein sequences (FIGS. 6 and 7). Conceptual translations from diverse strains indicate inteins of conserved sequence although some variation is apparent. The variation is more obvious in some strains. The variation is also more obvious in some regions, particularly the internal region. However, all of the proteins appear to encode putatively active inteins. The variation present at the DNA level is mostly restricted to changes in the third codon position both within the intein and the PRP8. There are additional numerous differences in the in the intron of the PRP8 gene.

TABLE 1

| | PHLS/ | | | Year of |
|---|---|---|---|---|
| No. | Bristol, UK | Isolated in: | Serotype | isolation |
| 1 | 3995 | Uganda | A(probably) | |
| 2 | 3997 | Uganda | A(probably) | |
| 3 | 8006 | Uganda | A(probably) | |
| 4 | 8071 | Thailand | A(probably) | |
| 5 | 8077 | Thailand | A(probably) | |
| 6 | 8104 | Thailand | A(probably) | |
| 7 | 8138 | UK | A(probably) | 1997 |
| 8 | 8237 | UK | A(probably) | 1998 |
| 9 | 8407 | UK | A(probably) | 1998 |
| 10 | 8408 | UK | A(probably) | 1999 |
| 11 | 8468 | UK | A(probably) | 2000 |
| 12 | 8565 | UK | A(probably) | 2001 |
| | IUM/Milan, | | | Year of |
| No. | Italy | Isolated in: | Serotype | isolation |
| | 86-0912 | | A | 1986 |
| | 87-3588 | | A | |
| | 89-6538 | | A | |
| | 90-2798 | | A | |
| | 91-6422 | | A | |
| | 91-4437 | | A | |
| | 91-5650 | | A | |
| | 92-4755 | | A | |
| | 94-3443 | | A | |
| | 94-5982 | | A | 1994 |
| | 88-3921 | | D | 1988 |
| | 89-4478 | | D | |
| | 89-0469 | | D | |
| | 90-2877 | | D | |
| | 91-6367 | | D | |
| | 92-6093 | | D | |
| | 93-0323 | | D | |
| | 93-1545 | | D | |
| | 93-3231 | | D | |
| | 94-0047 | | D | 1994 |
| | | | | Year of |
| No. | | Isolated in: | Serotype | isolation |
| | JEC21 | 1^{st} Sequencing | D | |
| | | strain | | |
| | CBS132 | Type strain/ | D | <1940 |
| | Aka: 3511 | Italy | | |
| | H99 | New strain for | A | |
| | | sequencing | | |
| | | | Molecular | Year of |
| | Name | Country | Type | isolation |
| 1 | WM 148 | Australia | VN I | 1989 |
| 2 | WM 626 | Australia | VN II | 1993 |
| 3 | WM 628 | Australia | VN III | 1988 |
| 4 | WM 629 | Australia | VN IV | 1987 |
| 5 | WM 179 | Australia | VG I/gattii | 1993 |
| 6 | WM 178 | Australia | VG II/gattii | 1991 |
| 7 | WM 175 | Australia | VG III/gattii | 1991 |
| 8 | WM 779 | South Africa | VG IV/gattii | 1994 |
| 9 | WM02.98 | Australia | VG II/gattii | 1985 |
| 10 | WM1249 | PNG | VG I/gattii | 1991 |
| 11 | WM728 | USA | VG III/gattii | 1992 |
| 12 | M27055 | South Africa | VG IV/gattii | 1996 |

### EXAMPLE 3

In-frame fusions of the CnePRP8 intein were made to ‘α peptide exteins’ carried by the expression plasmid pUC19 (AmpR) by restriction and ligation. The intein DNA was generated by polymerase chain reaction and pUC19 DNA was obtained commercially from New England Biolabs, USA. These constructs were transformed into E.coli DH5□Lac-) on Ampicillin selective medium. The transformed cells were able to grow on this medium as they had aquired ampicillin resistance. Plasmids were isolated from these cultures and sequenced. It was confirmed that they carried plasmid pCCNe in which intein sequences had been inserted into the pUC19 at the intended site in the expected inframe orientation. The pCCne colonies were blue in the presence of X-gal and were able to grow when lactose was the sole carbon source.

According to Sambrook et al., (Sambrook, J., MacCallum, P., David R. (2000) Molecular Cloning: A Laboratory Manual. Third edition. Cold Spring Harbor Laboratory Press) an insertion of this size of CnePRP8 would prevent α peptide complementation of β-galactosidase activity. The activity of this enzyme in bacteria containing pCCne indicates that the intervening protein sequence (the intein) has been excised from the precusor protein leaving a functional α peptide. This indicates the intein is active in this bacterial host and at this site in the α peptide coding sequence. If the intein function was blocked the cultures would grow white on X-gal and be unable to grow on minimal medium+lactose.

Similarly, In-frame fusions of the modified pRCne CnePRP8 intein were made to the ‘α peptide exteins’ carried by the expression plasmid pUC19 (AmpR) by restriction and ligation. The intein DNA was generated by polymerase chain reaction in which one primer was altered. The resulting sequence encoded an Arginine in place of a critical Cysteine at the N-terminus of the intein. The constructs were generated by restriction and ligation and transformed into E.coli DH5□Lac-) on Ampicillin selective medium. The transformed cells were able to grow on this medium as they had aquired ampicillin resistance. Plasmids were isolated from these cultures and sequenced. It was confirmed that they carried plasmid pRCNe in which mutant intein sequences had been inserted into the pUC19 at the intended site in the expected inframe orientation. The pRCne colonies were white in the presence of X-gal and were not able to grow when lactose was the sole carbon source.

The lack of activity of this enzyme in bacteria containing pRCne indicates that the intervening protein sequence (the intein) cannot be excised from the precusor protein resulting in a non-functional α peptide. The behaviour of pRCne transformed straind simulates the behaviour that would occur in strains in which the intein was blocked/inhibited by drugs.

### EXAMPLE 4

The intein peptide reporter construct may be grown in the presence and absence of an agent (usually a drug to be tested) at one, or varying concentrations of the drug and their phenotypes are scored at different drug concentrations. A drug might be for example zinc salts. Mills and Paulus (2001, Reversible inhibition of protein splicing by zinc ion. J Biol Chem. 276(14):10832-8) taking advantage of recently developed in vitro systems in which protein splicing occurs in trans (N- and C-exteins from different proteins) to assay for protein-splicing inhibitors, discovered that low concentrations of Zn(2+) inhibited splicing mediated both by the RecA intein from Mycobacterium tuberculosis and by the naturally split DnaE intein from Synechocystis sp. PCC6803. 90% inhibition was achieved by 0.2 mM zinc (6parts/million), a relatively low concentration. The inhibition of intein processing by zinc salts in vivo would be tested by inoculating DH5a carrying pCCne into one series of broths and as a control DH5a carrying pUC19 into another series of broths. Zinc salts would be added to the broths at various concentrations (0/0.2/2/20 mM). Intein inhibition would be inferred if the growth of the DH5a/pCCne cultures was inhibited at a zinc concentration that failed to inhibit the DH5a/pUC 19 culture.

### INDUSTRIAL APPLICATION

Inteins are not known from any mammalian gene, despite the very extensive amount of available sequence data. Accordingly, identified inteins are potential molecular targets with regard to the treatment of severe microbial infection. The administration of targeting drugs affecting the function of an intein (i.e. intein splicing inhibitors or antagonists) would arrest growth of the microbial pathogen without causing deleterious adverse effects in a patient (6). The concept of intein proteolytic inhibition is, in many ways, analogous to the aspartic protease inhibitor drug regime administered to HIV/AIDS patients.

Cryptococcus neoformans is one of the principal yeast pathogens of humans. It can cause incurable, frequently fatal, infections and has become especially significant as a predominant secondary pathogen associated with pandemic AIDS/human immunodeficiency virus infection. The most common clinical manifestation is chronic meningitis, which may be accompanied by lesions on the skin and lungs.

Cne PRP8 is only the second (non-allelic) eukaryotic nuclear intein to be identified. This yeast nuclear intein may be derived from inteins that were present in the genome of the archaeal component of the earliest eukaryotic organisms. It is also very distantly related to other inteins such as the VMA inteins from Ascomycete yeasts, and may therefore have distinctive sensitivity to inhibitory drugs and antagonists.

The PRP8 gene product is one of the most highly conserved proteins known (11), comprising the core of the spliceosome. The PRP8 gene product is an indispensable component of the spliceosome and therefore essential for cell viability. Loss of PRP8 function would result in an inability to process introns from all mRNA transcripts. PRP8 would be needed in very large amounts during rapid growth. Every intron in every message has to be removed before the mRNA can be active. Conversely in stationary phase there is minimal need for new mRNA and therefore minimal need for new PRP8. Therefore, PRP8 production represents a critical control point. Accordingly, the intein Cne PRP8 has a number of utilities in the field of medicine. In particular, this intein has value as a molecular target for therapeutic intervention.

For example, an intein or fragment of the invention may be used in drug screening programmes to select anti-intein agents (inhibitors or antagonists) useful in the treatment of microbial infections, especially cryptococcal infections in patients having AIDS or infected with HIV. A suitable method for screening an agent having intein inhibition activity is taught above and, for example in U.S. Pat. No. 5,795,731 (6). Minimal protein splicing elements such as Cne PRP8-Cn3511 which lack an endonuclease domain but have conserved sequence blocks at each end and/or polypeptides derived from Cne PRP8 may also be useful in the preparation of medicaments and/or in the development of therapeutic agents. For example, if an agent were to inhibit the proteolytic cleavage and/or splicing of the intein from the precursor protein, no active PRP8 gene product would be formed. The structure of inteins is well characterised including that of the yeast intein Sce VMA. The mode of action of inteins is also well understood at the molecular level. The system is therefore suitable for the development of appropriate inhibitory antagonists or inhibitors.

Those persons skilled in the art will understand that the above description is provided by way of illustration only and that the invention is not limited thereto.

### REFERENCES

1. Perler, F. B., Davis, E. O., Dean, G. E., Gimble, F. S., Jack, W. E., Neff, N., Noren, C. J Thorner, J., and Belfort, M. (1994) Protein splicing elements:inteins and exteins-a definition of terms and recommended nomenclature. Nucleic Acids Res., 22, 1125-1127.

2. Perler, F. B., Olsen, G. J., and Adam, E. (1997) Compilation and analysis of intein sequences. Nucleic Acids Res., 25, 1087-1093.

3. Kane, P. M., Yamashiro, C. T., Wolczyk, D. F., Neff, N., Goebl, M. and Stevens, T. H. (1990) Protein splicing converts the yeast TFP1 gene product to the 69-kD subunit of the vacuolar adenosine triphosphate. Science, 250, 651-657.

4. Gu, H. H., Xu, J., Gallagher, M. and Dean, G. E. (1993) Peptide splicing in the vacuolar ATPase subunit A from Candida tropicalis. J. Biol. Chem. 268, 7372-7381.

5. Gimble, F. S. and Thorner, J. (1992) Homing of a DNA endonuclease gene by meiotic gene conversion in Saccharomyces cerevisiae. Nature, 357, 301-306.

6. U.S. Pat. No. 5,795,731 dated 26 Aug. 1996 in the name of Health Research Incorporated.

7. Philippsen, P., Stotz, A., and Scherf, C. (1991) DNA of Saccharomyces cerevisiae. Methods Enzymol., 194, 169-182.

8. Thompson, J. D., Gibson, T. J., Plewniak, F., Jeanmougin, F., and Higgins, D. G. (1997) The CLUSTALX windows interface:flexible strategies for multiple sequence alignment aided by quality analysis tools. Nucleic Acids Res. 25, 4876-4882.

9. Galtier, N., Gouy, M., and Gautier, C. (1996) SEAVIEW and PHYLO_WIN: two graphic tools for sequence alignment and molecular phylogney. Comput. Appl.Biosci. 12, 543-548.

10. Beggs, J. D., Tiegelkamp, S., and Newman, A. J. (1995) The role of PRP8 protein in nuclear pre-mRNA splicing in yeast. J.Cell Sci. Suppl., 19, 101-5

11. Luo, H. R., Moreau, G. A., Levi, N., and Moore, M. J. (1999) The human PRP8 protein is a component of both U2- and U12-dependent spliceosomes. RNA, 5, 893-908.

12. Sambrook, J., MacCallum, P., David R. (2000) Molecular Cloning: A Laboratory Manual. Third edition. Cold Spring Harbor Laboratory Press

All references in this listing are in the text are incorporated herein by reference.

1. An isolated intein obtainable from Cryptococcus neoformans or a functionally equivalent, or functionally altered, fragment or variant thereof.

2. An intein, as claimed in claim 1 which can be isolated from C. neoformans strain Cn3511 on deposit at the American Type Culture Collection, Maryland, USA, under accession number ATCC 32045.

3. An intein as claimed in claim 1 or claim 2 which is obtainable from the C. neoformans PRP8.

4. An intein as claimed in any one of claims 1 to 3 wherein the intein comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, and SEQ ID NO:8.

5. An intein having the amino acid sequence of SEQ ID NO:1.

6. An intein which is a functionally equivalent variant or fragment of an intein as claimed in any one of claims 1 to 5.

7. An intein which is obtainable from an organism other than Cryptococcus neoformans and which is a functionally equivalent, or functionally altered, variant or fragment of an intein as claimed in any one of claims 1 to 6.

8. An isolated intein which has an amino acid sequence which has greater than about 35% identity with the sequence of SEQ ID NO:1.

9. An intein as claimed in claim 8 which has greater than 50% identity with the sequence of SEQ ID NO:1.

10. An intein as claimed in claim 8 or claim 9 which has greater than about 70% identity with the sequence of SEQ ID NO:1.

11. An intein as claimed in any one of claims 8 to 10 which has greater than about 80% identity with the sequence of SEQ ID NO:1.

12. An intein as claimed in any one of claims 8 to 11 which has greater than 90% identity with the sequence of SEQ ID NO:1.

13. An intein as claimed in any one of claims 8 to 12 which has greater than 95% identity with the sequence of SEQ ID NO:1.

14. An isolated nucleic acid molecule encoding an intein as claimed in any one of claim 1 to 13.

15. An isolated nucleic acid molecule which encodes an intein which is part of the genome of C. neoformans strain Cn 3511, on deposit at American Type Culture Collection, Maryland, USA, under accession number ATCC 32045.

16. A nucleic acid molecule as claimed in claim 15 which is obtainable from the C. neoformans PRP8 gene.

17. A nucleic acid molecule as claimed in any one of claims 14 to 16 which comprises a nucleic acid sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, and SEQ ID NO:17.

18. A nucleic acid molecule as claimed in claim 17 which has the nucleic acid sequence of SEQ I D NO:9.

19. A vector which includes a nucleic acid molecule as claimed in any one of claims 14 to 18.

20. A host cell which is capable of expressing a nucleic acid molecule as claimed in any one of claims 14 to 18.

21. A host cell which is transformed with a vector of claim 19.

22. A composition which comprises an intein as claimed in any one of claims 1 to 13.

23. An organism, in substantially pure form, which includes a nucleic acid molecule of any one of claims 14 to 18 and is capable of expressing an intein as claimed in any one of claims 1 to 13.

24. An intein as claimed in any one of claims 1 to 13 for use in medicine.

25. An intein as claimed in claim 24 wherein the use is as a target for testing agents for antimicrobial activity.

26. A protein including an intein as claimed in any one of claims 1 to 13.

27. A protein as claimed in claim 26 wherein the protein comprises an intein as claimed in any one of claims 1 to 13 by N- and C-terminal exteins.

28. A protein as claimed in claim 27 wherein the N- and C-terminal exteins comprise the protein C. neoformans PRP8.

29. A protein as claimed in claim 27 wherein the N- and C-terminal exteins comprise a reporter protein.

30. A protein comprising a binding protein portion, an intein as claimed in any one of claims 1 to 13, and a reporter protein portion.

31. A protein as claimed in claim 30 wherein the intein separates the binding protein portion and the reporter protein portion.

32. A protein as claimed in any one of claims 29 to 31 wherein the reporter protein is selected from an enzymatic assay protein a protein conferring antibiotic resistance, a protein providing a direct colorimetric assay, or a protein assayable by in vivo activity.

33. A protein as claimed in claim 32 wherein the reporter protein is selected from the group consisting of thymidylate synthase, β-galatosidase, galactokinase, alkaline phosphotase, β-lactamase, orotic acid decarboxylase, luciferase, and green fluorescent protein.

34. A protein as claimed in any one of claims 26 to 33 which is a fusion protein.

35. An isolated nucleic acid molecule which encodes a protein as claimed in any one of claims 26 to 33.

36. A method for producing a protein, the method comprising subjecting a protein as claimed in any one of claims 26 to 33 to cleavage conditions.

37. A method for screening an agent for antimicrobial activity against a microorganism, the microorganism having an intein of any one of claims 1 to 13 in a gene encoding a protein which facilitates growth of the microorganism, the method comprising detecting inhibition of said intein, which comprises:
(a) preparing recombinant clones of an inducible expression vector containing: (i) an altered reporter gene comprising a silent restriction site within a reporter gene, and (ii) said intein;
(b) detecting production of extein product of said intein by said recombinant clones in the presence of said agent;
wherein reduced production of said extein product indicates inhibition of said intein, and antimicrobial activity of said agent against said microorganism.

38. A method for screening an agent for antimicrobial activity against a micmoorganism, the microorganism having an intein of any one of claims 1 to 13 in a gene encoding a protein which facilitates growth of said microorganism, the method comprising detecting inhibition of said intein by monitoring intein function, which comprises:
(a) creating a silient restriction site within a reporter gene which results in an altered reporter gene;
(b) cloning said altered reporter gene into an inducible expression vector;
(c) cloning said intein into said inducible expression vector containing said altered reporter gene to generate recombinant clones; and
(d) detecting the production of extein product of said intein by said recombinant clones in the presence of said agent;
wherein reduced production of said extein product indicates inhibition of said intein, and antimicrobial activity of said agent against said microorganism.

39. A method as claimed in claim 37 or claim 38 wherein the protein is C. neoformans PRP8.

40. A method as claimed in any one of claims 37 to 39 wherein the intein further comprises an additional distal amino acid residue selected from the group consisting of cysteine, serine and threonine.

41. A method as claimed in any one of claims 37 to 40 wherein the reporter gene is β-galactosidase.

42. A method as claimed in any one of claims 37 to 41 wherein the microorganism is selected from the group consisting of C. neoformans, E. coli and Saccharomyces.

43. A method as claimed in any one of claims 37 to 42 wherein the inducible expression vector is PUC19.

44. A method as claimed in any one of claims 32 to 43 wherein detection of extein production is achieved by a method selected from the group consisting of phenotype characterisation, protein characterisation, tryptic peptide mapping and mass spectroscopy.

45. A method as claimed in claim 44 wherein detection of extein production is achieved by phenotype characterisation.

46. A diagnostic kit comprising an intein as claimed in any one of claims 1 to 13, or a protein as claimed in any one of claims 26 to 34 or primers therefore.

47. A kit as claimed in claim 46 which is a PCR assay kit.


SEQUENCE LISTING (17 sequences)

SEQ ID NO: 1 - protein, 172 aa, Cryptococcus neoformans
CLQNGTRLLRADGSEVLVEDVQEGDQLLGPDGTSRTASKIVRGEERLYRIKTHEGLEDLVCTHNHILSMYKERSGSERAHSPSADLSLTDSHERVDVTVDDFVRLPQQEQQKYQLFRSTASVRHERPSTSKLDTTLLRINSIELEDEPTKWSGFVVDKDSLYLRHDYLVLHN

SEQ ID NO: 2 - protein, 172 aa, Cryptococcus neoformans; identical to SEQ ID NO: 1

SEQ ID NO: 3 - protein, 171 aa, Cryptococcus neoformans
CLQNGTRLLRADGSEVLVEDVQEGDQLLGPDGTSRTASKIVRGEERLYRIKTHEGLEDLVCTHNHILSMYKERFGREGAHSPSAGTSLTESHERVDVTVDDFVRLPQQEQQKYKLFRSTDFVRREQPSASKLATLLHINSIELEEEPTKWSGFVVDKDSLYLRYDYLVLHN

SEQ ID NO: 4 - protein, 171 aa, Cryptococcus neoformans
CLQNGTRLLRADGSEVLVEDVQEGDQLLGPDGTSRTASKIVRGEERLYRIKTHEGLEDLVCTHNHILSMYKERFGREGAHSPSAGTSLTESHERVDVTVHDFVRLPQQEQQKYKLFRSTDSVRREQPSASKLATLLHINSIELEEEPTKWSGFVVDKDSLYLRYDYLVLHN

SEQ ID NO: 5 - protein, 171 aa, Cryptococcus neoformans; identical to SEQ ID NO: 3

SEQ ID NO: 6 - protein, 171 aa, Cryptococcus neoformans; identical to SEQ ID NO: 3

SEQ ID NO: 7 - protein, 171 aa, Cryptococcus neoformans; identical to SEQ ID NO: 3

SEQ ID NO: 8 - protein, 170 aa, Cryptococcus neoformans
CLQNGTRLLRADGSEILVEDVQDGDQLLGPDGTSRTASKIVRGEERLYRIKADELEDLVCTHNHILSLYKERSGSEQDPSPSTDLSSTDSYERVDVTVDDFVRLPQQEQQKYRLFRSTGFKRADQPSTSSLATLLHIMSIQLEEKPTKWSGFVVDKDSLYLRHDYLVLHN

SEQ ID NO: 9 - DNA, 516 bp, Cryptococcus neoformans
tgtcttcagaatggtactcgtcttctccgtgccgatggctctgaggtccttgtggaagatgttcaggagggcgatcaacttcttggtcccgatggaacgagcaggacggcgagcaagattgttcgcggcgaagagcgtctctaccgtatcaaaacccatgaggggctcgaagatcttgtctgtacccataaccacatcctttctatgtataaagaaaggtctggttcggagcgagctcattctcctagtgccgacctcagcctcacagacagccatgagagagtcgatgtgactgtcgatgactttgtccgccttcctcaacaagagcaacagaagtatcagcttttccgttcaactgcttctgtgcgacacgagcgaccatccacttctaaattagacaccaccttgttacgcatcaattctatcgagcttgaggacgagcctacgaagtggtccggttttgtggttgacaaagacagtctttatcttcgtcatgactatttggtattgcacaac

SEQ ID NO: 10 - DNA, 516 bp, Cryptococcus neoformans; identical to SEQ ID NO: 9

SEQ ID NO: 11 - DNA, 513 bp, Cryptococcus neoformans
tgtctgcagaatggtactcgtcttctccgtgccgatggctctgaggtccttgtggaagatgttcaggagggcgatcaacttcttggtcccgatggaacgagcaggacggcgagcaagattgttcgcggcgaagagcgtctctatcgtatcaaaacccatgagggactcgaagatctggtctgtacccataaccacatcctttctatgtataaagaaaggtttggtcgggagggagctcattctcccagtgccggcaccagcctcacagagagccatgagagagtcgatgtgactgtcgatgactttgtccgtcttcctcaacaagagcaacagaagtataagcttttccgttcaactgattttgtgcgacgcgaacaaccctccgcttctaaattagccaccttgttacacatcaattctatcgagcttgaggaggagcctacgaagtggtccggttttgtggttgacaaagacagtctttatcttcgttatgactatttggtactgcacaac

SEQ ID NO: 12 - DNA, 513 bp, Cryptococcus neoformans
tgtttgcagaatggtactcgtcttctccgtgccgatggctctgaggtccttgtggaagatgttcaggagggcgatcaacttcttggtcccgatggaacgagcaggacggcgagcaagattgttcgcggcgaagagcgtctctatcgtattaaaacccatgagggactcgaagatctggtctgtacccataaccacatcctttctatgtataaagaaaggtttggtcgggagggagctcattctcccagtgccggcaccagcctcacagagagccatgagagagtcgatgtgactgtccatgactttgtccgtcttcctcaacaagagcaacagaagtataagcttttccgttcgactgattctgtgcgacgcgaacaaccctccgcttctaaattagccaccttgttacacatcaattctatcgagcttgaggaggagcctacgaagtggtccggttttgtggttgacaaagacagtctttatcttcgttatgactatttggtactgcacaac

SEQ ID NO: 13 - DNA, 513 bp, Cryptococcus neoformans
tgtctgcagaatggtactcgtcttctccgtgccgatggctctgaggtccttgtggaagatgttcaggagggcgatcaacttcttggtcccgatggaacgagcaggacggcgagcaagattgttcgcggcgaagagcgtctctatcgtattaaaacccatgagggactcgaagatctggtctgtacccataaccacatcctttctatgtataaagaaaggtttggtcgggagggagctcattctcccagtgccggcaccagcctcacagagagccatgagagagtcgatgtgactgtcgatgactttgtccgtcttcctcaacaagagcaacagaagtataagcttttccgttcgactgattttgtgcgacgcgaacaaccctccgcttctaaattagccaccttgttacacatcaattctatcgagcttgaggaggagcctacgaagtggtccggttttgtggttgacaaagacagtctttatcttcgttatgactatttggtactgcacaac

SEQ ID NO: 14 - DNA, 513 bp, Cryptococcus neoformans
tgtttgcagaatggtactcgtcttctccgtgccgatggctctgaggtccttgtggaagatgttcaggagggcgatcaacttcttggtcccgatggaacgagcaggacggcgagcaagattgttcgcggcgaagagcgtctctatcgtattaaaacccatgagggactcgaagatctggtctgtacccataaccacatcctttctatgtataaagaaaggtttggtcgggagggagctcattctcccagtgccggcaccagcctcacagagagccatgagagagtcgatgtgactgtcgatgactttgtccgtcttcctcaacaagagcaacagaagtataagcttttccgttcgactgattttgtgcgacgcgaacaaccctccgcttctaaattagccaccttgttacacatcaattctatcgagcttgaggaggagcctacgaagtggtccggttttgtggttgacaaagacagtctttatcttcgttatgactatttggtactgcacaac

SEQ ID NO: 15 - DNA, 513 bp, Cryptococcus neoformans; identical to SEQ ID NO: 13

SEQ ID NO: 16 - DNA, 510 bp, Cryptococcus neoformans
tgtctgcagaatggtacccgtcttctccgtgctgatggttccgagattcttgtggaagatgttcaagacggcgatcagcttcttggtcccgatggaacgagcaggacagcgagcaagatcgttcgcggtgaagagcgtctctatcgtatcaaagctgatgaactcgaagatctggtctgtacacacaatcacatcctctcattgtataaggaaaggtctggctcggagcaagatccttctcctagtaccgacctcagctcgacggatagctatgagagagttgacgtgactgtcgatgactttgtccgccttcctcaacaagagcaacagaagtatcggcttttccgttcaactggttttaagcgagccgatcagccttccacttcttcattagccaccttgttacatatcatgtctatccagctggaggaaaagcctacaaagtggtccggttttgtggtcgacaaagacagcctttatctccgtcatgactatttggtattacacaac

SEQ ID NO: 17 - DNA, 516 bp, Cryptococcus neoformans
tgtctgcagaatggtactcgtcttctccgtgccgatggctctgaggtccttgtggaagatgttcaggagggcgatcaacttcttggtcccgatggaacgagcaggacggcgagcaagattgttcgcggcgaagagcgtctctaccgtatcaaaacccatgaggggctcgaagatcttgtctgtacccataaccacatcctttctatgtataaagaaaggtctggttcggagcgagctcattctcctagtgccgacctcagcctcacagacagccatgagagagtcgatgtgactgtcgatgactttgtccgccttcctcaacaagagcaacagaagtatcagcttttccgttcaactgcttctgtgcgacacgagcgaccatccacttctaaattagacaccaccttgttacgcatcaattctatcgagcttgaggacgagcctacgaagtggtccggttttgtggttgacaaagacagtctttatcttcgtcatgactatttggtattgcacaac